(19) United States
(12) Patent Application Publication
(10) Pub. No.: US 2004/0115285 A1
(43) Pub. Date: Jun. 17, 2004

(54) Method of normalizing glucose levels in blood of patients with diabetes mellitus by oral administration of proanthocyanidins containing plant extracts

(76) Inventor: Peter Rohdewald, Muenster (DE)

Correspondence Address: GIBBONS, DEL DEO, DOLAN, GRIFFINGER & VECCHIONE, 1 RIVERFRONT PLAZA, NEWARK, NJ, 07102-5497, US

(21) Appl. No.: 10/319,334

(22) Filed: Dec. 13, 2002

(51) Int. Cl. A61K 38/28; A61K 35/78; A61K 31/353

(52) U.S. Cl. 424/725; 514/3; 514/456

(57) ABSTRACT

A method for reducing blood glucose levels in patients with diabetes mellitus using as pharmaceutically effective agent a plant extract containing a high percentage of proanthocyanidins is provided. The method includes administering to hyperglycemic patients a proanthocyanidins containing plant extract in preparations suitable for oral intake in a dosage that is therapeutically effective to decrease elevated glucose levels or elevated glycosylated hemoglobin levels in blood of diabetics.

### FIELD OF THE INVENTION

The present invention relates to the use of plant extracts containing proanthocyanidins to lower the elevated blood glucose levels of patients with diabetes mellitus.

### BACKGROUND OF THE INVENTION

Diabetes Mellitus

Diabetes mellitus is a wide spread disease affecting a substantial part of the population. The most obvious sign of diabetes mellitus is an abnormally elevated concentration of glucose in the blood. The high glucose level signals a pathological condition of metabolism, affecting the metabolism of carbohydrates, lipids and proteins. Diabetes mellitus is characterized by hyperglycemia resulting from impaired insulin secretion and is associated with a host of late complications including retinopathy, nephropathy, atherosclerotic coronary and peripheral arterial disease, and peripheral and autonomic neuropathies. There are two main types of diabetes mellitus. In Type I, or insulin dependent diabetes (also known as juvenile diabetes), the insulin-secreting cells in the pancreas are destroyed and insulin production ceases almost completely. Type II, or non-insulin-dependent diabetes (adult-onset diabetes), is usually marked by gradual onset wherein insulin is produced, but not in sufficient quantity to fully metabolize blood glucose. Both types result in an abnormally high level of glucose in the blood, which, if left uncorrected, can cause coma and death.

To overcome the lack of insulin, substitution of insulin is a logical and successful way to treat type I diabetes. Conventional insulin treatment for diabetes includes one or two injections per day of intermediate-acting insulin, with or without smaller added doses of rapid-acting insulin in the same syringe. Treatment of type II diabetes starts with diet. In a more intensive treatment of type II diabetes sulphonylurea derivates, such as, for example, tolbutamide, or biguanidines, such as metformin, or alpha glucosidase inhibitors, such as acarbose, and finally insulin are used. More recently, glitazons have been added to the therapy as insulin sensitizers which reinforce or start the action of insulin.

There are many side effects associated with conventional treatments of diabetes mellitus. For example, injected at regular intervals, insulin shots cause discomfort in patients. Complications of insulin treatment include severe hypoglycemia, local allergic reactions, generalized insulin allergy, immunologic insulin resistance and local fat cell atrophy or hypertrophy. Sulphonylureas are limited in their success rate by the secretory capability of the β-cells of the pancreas; metformin has several contraindications and glitazones cause weight increase in patients. Hypoglycemia is the most important complication of sulfonylurea treatment. Sulfonylurea-induced hypoglycemia can be severe and may last or recur for days after treatment is stopped.

Proanthocyanidins

Proanthocyanidins represent a group of plant polyphenols found in roots, barks and fruits with an astringent taste. Proanthocyanidins include the subgroups of procyanidins and prodelphinidins. Proanthocyanidins are biopolymers composed of flavan subunits. Procyanidins are composed of catechin and epicatechin units, also called monomeric procyanidins. The structure of a trimer of proanthocyanidins is shown in FIG. 1.

Proanthocyanidins are extracted from plant material by conventional methods using solvents like water, ethanol or acetone or fluid carbon dioxide. The extracts are purified by solvent/solvent extraction, ultra filtration or chromatographic procedures. The purified extracts are concentrated by solvent evaporation, freeze drying or spray drying.

An extract from the bark of French maritime pine Pycnogenol®, distributed by Horphag Research, Switzerland contains 70-75% by weight proanthocyanidins and other flavanols such as catechin, epicatechin and taxifolin. Furthermore, the extract contains phenolic acids such as caffeic acid, ferulic acid, p-coumarinic acid and p-benzoic acid, which are all present in plants. Of these acids, some are combined with glucose, forming glucose esters or glucose ethers. The extract from pine barks and especially Pycnogenol® pine bark extract contains essentially condensed tannins and no hydrolysable tannins. Other proanthocyanidins rich extracts can be obtained from grape seeds, cones from cypress trees, cocoa beans or other plant materials.

One serious form of microangiopathy is the diabetic retinopathy. It has been reported that oral applications of extracts of proanthocyanidins slow down the progression of the retinopathy disease. (Schönlau and Rohdewald, Int. Ophthalmology, 2002, accepted for publication, Saracco and Estachy, Gaz. Med. France, 88: 2035 2038 (1981); Lagrue et al., Sem. Hop. Paris 57: 33 36 (1981)). It has also been reported that the symptoms of diabetic retinopathy, such as microbleedings or microaneurisms, have been ameliorated by supplementation with an extract containing proanthocyanidins, but there was no influence on glycemia or glucosuria found in diabetic patients. (Fromantin, Medicine interne, 16: 432-434 (1981)). Beneficial results in treating diabetic or other retinopathies with proanthocyanidins containing extracts have been attributed to sealing of leaky capillaries by proanthocyanidins, which cause strong protein binding thus preventing the outflow of blood or liquid out of leaky capillaries. As a result, the number of microbleedings in the retinal tissue is reduced, and the visual acuity of the patients can improve.

The lowering of glucose levels in diabetics after oral intake of proanthocyanidins has not been previously reported. The use of proanthocyanidins for treatment of diabetic retinopathy was based on the beneficial effect of proanthocyanidins on the capillary wall of blood vessels. That mechanism was demonstrated by experiments with hypertensive rats having weak capillaries because of chronic hypertension. The weak capillaries produced microbleeding when a slight vacuum was applied to the skin. Oral supplementation with a proanthocyanidins containing extract such as Pycnogenol® sealed the leaky walls of the capillaries within just one hour after oral administration of the extract to hypertensive rats. These experiments conducted with non-diabetic, hypertensive rats demonstrated the activity of a proanthocyanidin-containing extract in sealing leaky capillaries and thus normalizing pathologically low capillary resistanc. Accordingly, experiments with nondiabetic rats explain the mechanism of the action of proanthocyanidins in case of diabetic retinopathy as being due to sealing of leaky capillaries and not to lowering of blood glucose levels. (Gabor et al., Phlebologie, 22; 178-182, 1993) Thus, the unexpected finding that plant extracts containing proanthocyanidins did not only affect the capillary system but also decreased blood glucose levels in diabetics could not be deduced from the beneficial effects of Pycnogenol® found in case of diabetic retinopathy.

Other beneficial effects of proanthocyanidins containing extracts are due to their antioxidant activity which can fight the oxidative stress associated with diabetes, and the prevention of thrombus formation. Thrombus formation is a leading cause of death of diabetics. The inhibition of platelet aggregation by proanthocyanidins, which also inhibits thrombus formation, is another beneficial effect of proanthocyanidins containing extracts. A hypoglycemic effect in diabetic patients had not been previously observed together with the foregoing positive effects of proanthocyanidins.

U.S. Pat. No. 5,531,991 to Cheng, et al. describes the use of an alkaline aqueous extract from the roots of Polygonum multiflorum for treating hyperglycemia. Cheng et al. do not disclose the composition of that extract obtained from Polygonum multiflorum. However, a publication by Nonaka et al. describes an ethyl acetate extract from Polygonum multiflorum containing stilbene glycoside gallates and galloyl procyanidins (Nonaka et al., Stilbene glycoside gallates and proanthocyanidins from Polygonum multiflorum, Phytochemistry 21: 429 432 (1982)). It has not been reported that an alkaline extract of Polygonum multiflorum as described in the '991 reference contains the same constituents as the ethyl acetate extract described in Nonaka et al., namely galloylated stilbene glycosides and galloylated procyanidins. Neither U.S. Pat. No 5,531,991 nor the publication of Nonaka et al. describe an extract from Polygonum multiflorum containing proanthocyanidins or non-galloylated, free procyanidins useful to lower blood glucose levels in patients suffering from diabetes mellitus.

As used herein, “free procyanidins” refer to non-galloylated procyanidins that are not substituted with gallic acid, are not hydrolysable and are classified as condensed tannins (FIG. 1). In contrast, “galloylated procyanidins” are the result of esterification of procyanidins with gallic acid. The esterification with gallic acid changes the molecular weight of procyanidins, their redox potential and affinity to proteins and enzymes. Galloylated procyanidins belong to the group of hydrolysable tannins and, as discussed above, are physically and chemically different from condensed tannins (FIG. 4).

Thus far, no prior reference has described any hypoglycemic effect of a plant extract containing proanthcyanidins. There is therefore still a need to provide a method of reducing glucose levels in the blood of patients with diabetes mellitus that avoids the many side effects associated with conventional treatments of this disease. The unexpected finding that a plant extract containing proanthcyanidins is capable of lowering blood glucose and/or glycosylated hemoglobin levels when administered to patients having diabetes mellitus provides such a method.

### SUMMARY OF THE INVENTION

The present invention provides a method for reducing an elevated level of blood glucose or glycosylated hemoglobin in patients suffering from diabetes mellitus by orally administering a proanthocyanidins containing plant extract. In an aspect of the invention, the extract useful in the invention excludes extracts from Polygonum multiflorum.

In another aspect, the method of the invention further comprises providing the proanthocyanidins rich plant extract as functional food, food supplement or pharmaceutical preparations for oral administration to a patient in need. The proanthocyanidins containing plant extract could be administered in liquid, semisolid or solid form. The daily dosage is from about 20 to about 500 mg of the proanthocyanidins containing plant extract, depending on the body weight of the diabetic, calculated as the proanthocyanidins component of the extract.

The plant extracts useful in the method provided by the invention include a plant extract containing from about 30% to about 100% proanthocyanidins, preferably condensed tannins or non-galloylated procyanidins. In another aspect, the plant extracts useful to lower blood glucose levels upon administration to diabetic patients include a plant extract containing from about 60% to about 80% proanthocyanidins, preferably condensed tannins or non-galloylated procyanidins.

The description is not in any way intended to limit the scope of the present invention, but rather only to provide a working example of the presently preferred embodiments. The scope of the present invention will be pointed out in the appended claims.

### BRIEF DESCRIPTION OF THE DRAWINGS

For a better understanding of the present invention, reference is made to the following description and accompanying drawings, while the scope of the invention is set forth in the appended claims

FIG. 1 shows the principal structure of procyanidins.

FIG. 2 is a box plot of the differences of glucose concentrations in blood at the start of the trial to the values obtained during the observation period. (Glucose is shown at 0 weeks and at x weeks).

FIG. 3 is a box plot of the differences of HbA, concentrations in blood at the start of the trial to the values obtained during observation period. (HbA, is shown at 0 months and at x months.)

FIG. 4 shows the principal structure of galloylated procyanidins.

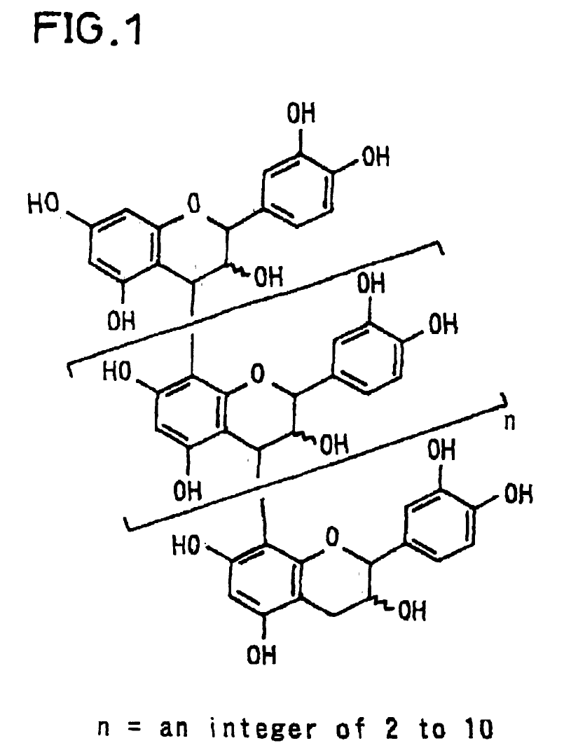

### DETAILED DESCRIPTION OF THE INVENTION

The present invention is directed to the discovery that a composition including plant extracts containing proanthocyanidins, and especially condensed tanins, upon administration to diabetic patients can lower pathologically high blood glucose concentrations. This discovery arose from a clinical study investigating the effects of a proanthocyanidins containing pine bark extract (Pycnogenol®), on diabetic patients with hypertension. The study was designed primarily to find out whether supplementation of diabetic patients with an extract containing proanthocyanidins would allow a reduction in the dose of antihypertensive drugs.

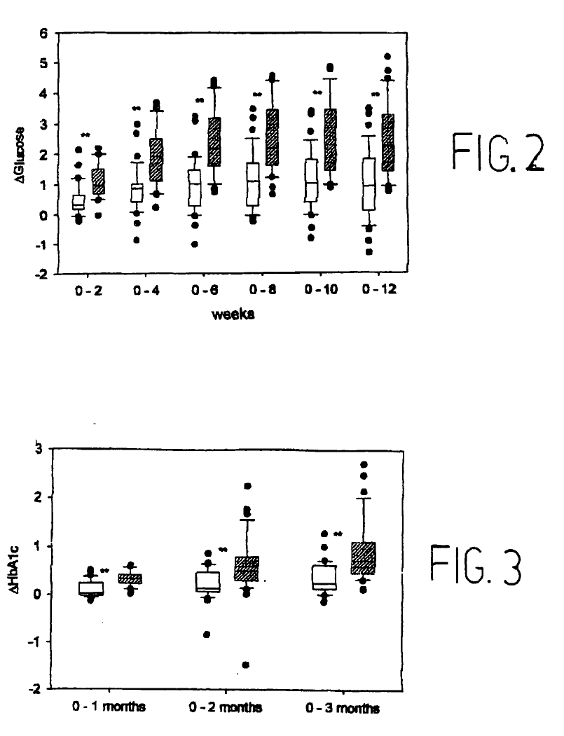

As illustrated in FIG. 2, by monitoring glucose concentrations among other parameters of clinical chemistry, it was unexpectedly found that glucose concentration levels in the blood of diabetic patients taking a proanthocyanidins containing plant extract decreased continuously over the period of supplementation with the plant extract. The decrease in blood glucose level observed in the patients treated with the plant extract was significantly higher as compared to patients taking only a placebo. In FIG. 2, results obtained by treating patients with placebo and with the proanthocyanidins-rich plant extract are shown as white and shaded boxes, respectively. The significance of differences between results obtained by administering a placebo or a plant extract containing proanthocyanidins to diabetic patients was calculated by Mann Whitney Rank Sum test with a Bonferroni correction: p<0.01.

Furthermore, as illustrated in FIG. 3, glucosylated hemoglobin HbA₁, the marker for permanently elevated glucose levels in blood, was also significantly lowered in patients taking the extract containing proanthocyanidins as compared to those taking only a placebo. In FIG. 3, results obtained by treating patients with placebo and with the proanthocyanidins-rich plant extract are shown as white and shaded boxes, respectively. The significance of differences between results obtained by administering placebo or an extract containing proanthocyanidins was calculated by Mann Whitney Rank Sum test with a Bonferroni correction: p<0.01.

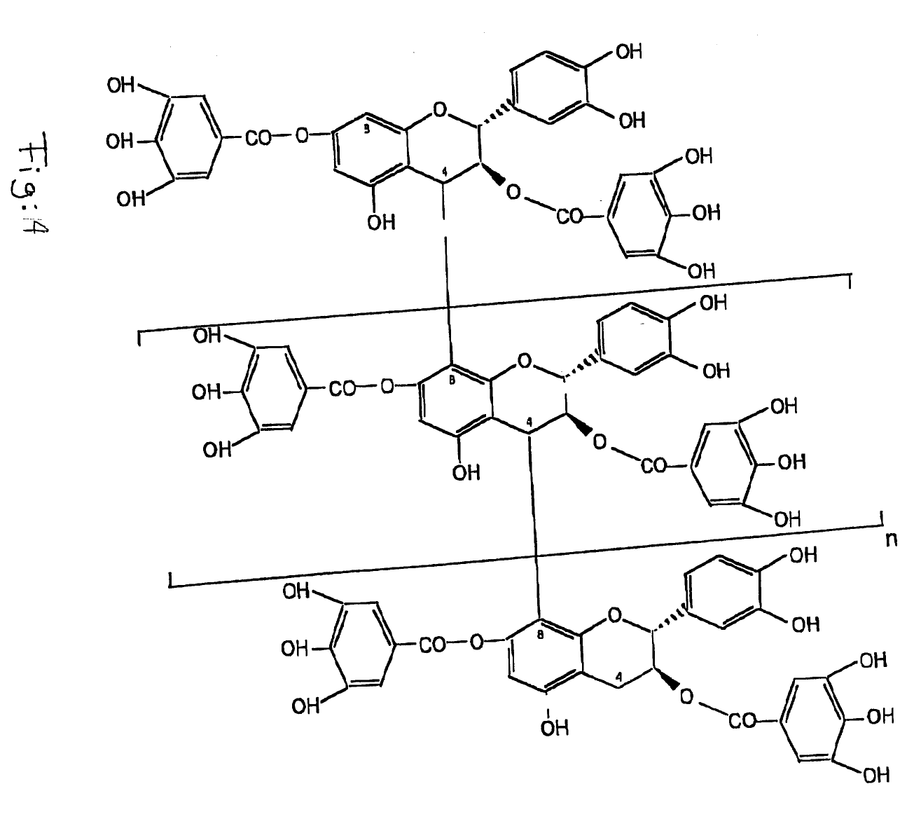

The extracts containing proanthocyanidins employed in the method of the invention can be produced, for example, from the French maritime pine bark or grape seeds. Proanthocyanidins include the subgroups of procyanidins and prodelphinidins. Procyanidins can be non-galloylated or galloylated as described and as to the latter as shown in FIG. 4.

The present invention relates to the use of a plant extract containing proanthocyanidins for treating a patient having elevated blood glucose level and/or elevated levels of glucosylated hemoglobin wherein the plant extract is the effective component.

The invention provides a method of orally administering preparations comprising a proanthocyanidins containing extract, in the form for example of capsules, tablets, or a liquid or powdery preparation.

Furthermore, the method of the invention further comprises administering other pharmaceutically active agents together with the proanthocyanidins containing plant extract to lower blood glucose levels. Other conventional hypoglycemic agents can be administered, for example such as those selected from the group consisting of exogenous insulin, a sulfonylurea, a biguanidine, a thiazolidione, a β₃-adrenoreceptor agonist or an α-glucosidase inhibitor. The conventional hypoglycemic agent can be administered simultaneously with, prior to or after orally administering the proanthocyanidins containing plant extract of the invention. The conventional hypoglycemic agents can be administered in an amount sufficient to lower an elevated level of blood glucose or glycosylated hemoglobins to a level that is lower than the level attained from orally administering the proanthocyanidins containing plant extract.

Besides the hypoglycemic agents described above, oral dosage forms useful in the method of the invention generally contain conventional excipients such as binder, disintegrates, lubricants and glydants. In addition, any of the conventional methods utilized in formulating these oral unit dosage forms can be utilized in accordance with this invention.

The present invention may be better understood by reference to the following non-limiting examples. These examples should in no way be construed, however, as limiting the broad scope of the invention.

### EXAMPLES

The proanthocyanidins used in the clinical studies described below are obtained from Pycnogenol® pine bark extract, an extract from the bark of the French maritime pine, produced and marketed by Horphag Research Limited.

### Example 1

57 patients were subjected to a clinical trial conducted in a completely randomized, double blind, placebo controlled study. 29 patients were getting placebo, 28 patients received a dose of 200 mg of concentrate of proanthocyanidins of French maritime pine bark extract (Pycnogenol® pine bark extract). Patients took the placebo or Pycnogenol® pine bark extract in addition to their normal medication. At intervals of two (2) weeks, the blood glucose of each patient was measured before breakfast by a specific commercial assay by using a glucoseoxidase peroxidase system. The results are shown in FIG. 2.

The decrease of blood glucose concentrations relative to the starting values was large over the whole period of the clinical trial following supplementation with the extract. Patients given the placebo showed a decrease of glucose levels that was statistically significantly smaller over the whole period of the study.

The results of this example demonstrate that blood glucose levels of diabetics which could not be lowered to normal values by conventional treatment were significantly lowered after the administration of an extract of French maritime pine bark.

### Example 2

The same group of patients as in example 1 was monitored at monthly intervals for concentrations of glycosylated hemoglobin (HbA₁) in blood. Hemoglobin reacts with glucose when glucose levels are high, forming a modified, stable hemoglobin containing glucose, wherein hemoglobin is chemically bound to glucose. As a result, HbA₁ can be found in blood over a long period of time and reflects the accumulated products of reaction between glucose and hemoglobin. Whereas the monitoring of glucose itself in blood reflects only the situation at the moment of blood sampling, HbA₁ values indicate whether glucose concentrations were normal or abnormally high over a period of 120 days before blood sampling.

FIG. 3 shows that the differences of HbA₁ values relative to starting values were statistically significantly larger following supplementation of diabetic patients with an extract containing proanthocyanidins as compared to those administered only a placebo. The differences increased continuously over the period of the trial suggesting that HbA₁ levels could be further decreased to normal values after a longer period of treatment.

The results of this example demonstrate that glucose concentrations in blood which could not be lowered to normal values by conventional treatment, were lowered significantly after administering to diabetic patients a proanthocyanidins containing extract. The proanthocyanidins containing extract used in examples 1 and 2 had a high content of proanthocyanidins of about 70%. The dose taken by patients was 200 mg daily of extract over a period of 3 months. The results indicate that a longer period of treatment and/or a higher daily dose could be even more effective, because there was still a further decrease of HbA₁ levels after 3 months. The supplementation with proanthocyanidins containing extract was performed as an addition to existing treatment using traditional Chinese medicine or hypoglycemic drugs.

All of the publications and references cited in the foregoing specification, are incorporated herein by reference as if set forth in full for all they disclose.

While the foregoing description and drawings represent the preferred embodiments of the present invention, it will be understood that various changes and modifications may be made without departing from the spirit and scope of the present invention.

We claim:

1. A method of reducing an elevated level of blood glucose or glycosylated hemoglobin in a hyperglycemic patient which comprises:
orally administering a proanthocyanidins containing plant extract to said hyperglycemic patient, said proanthocyanidins being in a therapeutically effective amount sufficient to lower said elevated level of blood glucose or glycosylated hemoglobin to a level less than said elevated level blood glucose or glucosylated hemoglobin.

2. The method of claim 1, wherein said proanthocyanidins containing plant extract is other than a plant extract derived from Polygonum multiflorum.

3. The method of claim 1, further comprising selecting said proanthocyanidins from the group consisting of non-galloylated procyanidins.

4. The method of claim 1, wherein said proanthocyanidins containing plant extract is in an oral dosage form comprising from about 20 mg to about 500 mg.

5. The method of claim 1, wherein said proanthocyanidins containing plant extract includes from about 30% to about 100% by weight proanthocyanidins.

6. The method according to claim 1, further comprising providing the proanthocyanidins containing plant extract as food, a food supplement or a medicinal composition.

7. The method according to claim 6, further comprising administering the proanthocyanidins containing composition in liquid, semisolid or solid form.

8. The method of claim 6, further comprising administering a hypoglycemic agent selected from the group consisting of exogenous insulin, a sulfonylurea, a biguanidine, a thiazolidione, a β₃-adrenoreceptor agonist, or an α-glucosidase inhibitor, wherein said hypoglycemic agent is administered simultaneously with, prior to or after orally administering said proanthocyanidins containing plant extract, said hypoglycemic agent in an amount sufficient to lower said elevated level of blood glucose or glycosylated hemoglobin to a level that is lower than the level attained from orally administering said proanthocyanidins containing plant extract.
